(19) United States
(12) Patent Application Publication
(10) Pub. No.: US 2026/0345214 A1
(43) Pub. Date: Nov. 5, 2026

(54) Edge-Mounted Modular Smoke Evacuation System for Surgical Applications

(71) Applicant: Surgical Safety Solutions Inc., Wilmington, DE (US)

(72) Inventors: Syed Khalid, Chicago, IL (US); Hamad Issam Farhat, Chicago, IL (US)

(21) Appl. No.: 19/197,247

(22) Filed: May 2, 2025

(51) Int. Cl. A61B 18/00

(52) U.S. Cl. CPC A61B 18/00; A61B 2218/008

(57) ABSTRACT

Example embodiments relate to edge-mounted modular smoke evacuation systems for surgical applications. One example embodiment includes a system. The system includes a surgical drape configured to be positioned adjacent to at least one side of a surgical site. The system also includes one or more flexible tubes removably attached to or positioned within the surgical drape. Each of the one or more flexible tubes includes a plurality of holes, and each of the one or more flexible tubes is positioned such that the plurality of holes are directed toward the surgical site. Each of the one or more flexible tubes terminates at a fitting. The system also includes a vacuum source connected to the respective fittings of the one or more flexible tubes and configured to evacuate fluid generated at the surgical site through the plurality of holes by establishing a pressure differential.

### BACKGROUND

During surgical procedures, especially those that involve electrocautery, laser ablation, or ultrasonic dissection, the generation of surgical smoke and other harmful gases is a persistent challenge. Surgical smoke can contain a spectrum of harmful substances, including volatile organic compounds, fine particulate matter, and potentially viable pathogens. Prolonged exposure can lead to respiratory irritation, ocular discomfort, carcinogenic risks, and transmission of infectious agents. The resulting reduction in visibility due to smoke particulate matter may also compromise surgical precision.

While the hazards of surgical smoke are widely recognized, widespread implementation of effective smoke evacuation strategies remains inconsistent. Several regulatory and advisory bodies in the United States, such as the Occupational Safety and Health Administration, the Centers for Disease Control and Prevention, and the National Institute for Occupational Safety and Health, urge the use of comprehensive smoke management protocols. However, current techniques often fail to meet the combined requirements of flexibility, cost-effectiveness, noise minimization, and efficient, at-source capture.

### SUMMARY

The embodiments herein address the above-identified problems, as well as other issues, by presenting a smoke evacuation system that may be integrated into or attached to a surgical drape.

In a first aspect, the disclosure describes a system. The system includes a surgical drape configured to be positioned adjacent to at least one side of a surgical site. The system also includes one or more flexible tubes removably attached to or positioned within the surgical drape. Each of the one or more flexible tubes includes a plurality of holes, and each of the one or more flexible tubes is positioned such that the plurality of holes are directed toward the surgical site. Each of the one or more flexible tubes terminates at a fitting. The system also includes a vacuum source connected to the respective fittings of the one or more flexible tubes and configured to evacuate fluid generated at the surgical site through the plurality of holes by establishing a pressure differential.

In a second aspect, the disclosure describes a method. The method includes removably attaching one or more flexible tubes to a surgical drape configured to be positioned adjacent to at least one side of a surgical site. Each of the one or more flexible tubes includes one or more holes. Each of the one or more flexible tubes is positioned such that the one or more holes are directed toward the surgical site. Each of the one or more flexible tubes terminates at a fitting. The method also includes connecting the one or more flexible tubes to a vacuum source using the respective fittings. The method also includes activating the vacuum source to establish a pressure differential. The method also includes evacuating fluid generated at the surgical site through the one or more holes of the one or more flexible tubes based on the pressure differential.

In a third aspect, the disclosure describes an apparatus. The apparatus includes a flexible tube comprising an exterior surface and an interior surface. The interior surface surrounds an interior chamber, and one or more holes are defined between the exterior surface and the interior surface of the flexible tube such that the interior chamber is fluidly connected to an exterior of the flexible tube. The flexible tube terminates at a fitting configured to connect to a vacuum source in order to evacuate fluid generated at a surgical site through the one or more holes via a pressure differential. The apparatus also includes an adhesive strip, one or more clips, or one or more mounts connected to the flexible tube and configured to removably attach the flexible tube to a surgical drape.

The foregoing summary is illustrative only and is not intended to be in any way limiting. In addition to the illustrative aspects, embodiments, and features described above, further aspects, embodiments, and features will become apparent by reference to the figures and the following detailed description.

### BRIEF DESCRIPTION OF THE FIGURES

FIG. 1A is an illustration of a smoke evacuation system, according to example embodiments.

FIG. 1B is an illustration of a smoke evacuation system with flexible tubes in a series configuration, according to example embodiments.

FIG. 1C is an illustration of a smoke evacuation system with flexible tubes in a parallel configuration, according to example embodiments.

FIG. 1D is an illustration of a smoke evacuation system with two vacuum sources, according to example embodiments.

FIG. 1E is an illustration of a flexible tube attached to a surgical drape via an adhesive layer, according to example embodiments.

FIG. 1F is an illustration of a flexible tube attached to a surgical drape via a clip, according to example embodiments.

FIG. 1G is an illustration of flexible tube integrated within a surgical drape, according to example embodiments.

FIG. 2A is an illustration of a flexible tube with evenly spaced holes, according to example embodiments.

FIG. 2B is an illustration of a flexible tube with variably spaced holes, according to example embodiments.

FIG. 2C is an illustration of a flexible tube, according to example embodiments.

FIG. 2D is a cross-sectional view of the flexible tube of FIG. 2C, according to example embodiments.

FIG. 2E is a cross-sectional view of a flexible tube indicating diameters of an exterior surface and an interior surface, according to example embodiments.

FIG. 2F is a cross-sectional view of a flexible tube including a hole with a fluted shape, according to example embodiments.

FIG. 2G is a cross-sectional view of a flexible tube including a hole with a straight shape, according to example embodiments.

FIG. 2H is a cross-sectional view of a flexible tube including a hole with an angled shape, according to example embodiments.

FIG. 2I is a cross-sectional view of a flexible tube including a lining, according to example embodiments.

FIG. 2J is a cross-sectional view of a flexible tube with an exterior surface including a flattened surface, according to example embodiments.

FIG. 3 is a flowchart of a method, according to example embodiments.

### DETAILED DESCRIPTION

Example methods and systems are described herein. Any example embodiment or feature described herein is not necessarily to be construed as preferred or advantageous over other embodiments or features. The example embodiments described herein are not meant to be limiting. It will be readily understood that certain aspects of the disclosed systems and methods can be arranged and combined in a wide variety of different configurations, all of which are contemplated herein.

Furthermore, the particular arrangements shown in the figures should not be viewed as limiting. It should be understood that other embodiments might include more or less of each element shown in a given figure. In addition, some of the illustrated elements may be combined or omitted. Similarly, an example embodiment may include elements that are not illustrated in the figures.

Herein, coordinate axes x, y, z are provided in the figures for reference. Other axes and/or orientations may be used in some embodiments. Generally, “longitudinal axis” refers to an axis that runs lengthwise down the greater length of a flexible tube along a center of the flexible tube.

Herein, reference is made to “smoke” or “surgical smoke” as the fluid evacuated by certain embodiments of this disclosure as an example and should not be construed as limiting. Other fluids, such as other gases or liquids, may be evacuated by various embodiments of this disclosure in addition to or instead of smoke or surgical smoke.

It is understood that, though “flexible tubes” are described throughout the present disclosure, example embodiments may also include rigid tubes. For example, materials used to fabricate the tubes described herein could be rigid plastics or metals. Further, even in embodiments where the tubes are “rigid,” such tubes may still be repositionable relative to a surgical drape or a surgical site (e.g., by a surgeon during surgery).

### 1. Overview

Current techniques for surgical smoke evacuation exhibit several shortcomings. Some techniques rely on stationary devices that generate air curtains or rely on distant suction. Such systems are often placed adjacent to the operating table and do not adapt to changes in the surgical drape configuration. This rigidity can obstruct surgical access, fail to maintain consistent smoke capture as the drape is repositioned, and potentially create escape points for smoke. Other techniques attempt to control smoke via ambient airflow or through ceiling-mounted units. However, this off-source technique is inefficient. The lack of immediate, localized capture increases the risk that smoke will diffuse into the operating room environment, reducing overall evacuation efficiency and necessitating higher suction power and energy usage.

Further, ceiling-mounted systems and large stand-alone evacuators can require significant infrastructural changes, capital investments, and dedicated personnel for operation or repositioning. Handheld suction devices, while flexible, demand continuous manual operation and can distract personnel from the primary surgical tasks. These constraints hinder broad implementation in a range of healthcare settings, from large hospitals to smaller outpatient clinics.

Many current techniques also produce disruptive noise due to turbulent airflow and high suction velocities. Excessive noise can inhibit communication between those working in the operating room and can, therefore, increase stress levels. In addition, the fluid dynamics of current techniques are often not optimized to ensure stable, laminar flow patterns, resulting in unpredictable capture efficiency and potential pressure fluctuations.

Example embodiments relate to a modular, edge-mounted smoke evacuation system that may be integrated within or attached to a surgical drape. Example systems may include one or more flexible tubes fitted with inward-facing perforations that capture smoke directly at the source. Further, example systems may integrate directly with existing operating room suction systems without requiring additional specialized equipment.

The embodiments herein offer several advantages over alternative techniques to smoke evacuation in surgical applications. One advantage is providing a modular and scalable design. The embodiments herein allow multiple tubes to be arranged in series or parallel to conform to varying surgical field sizes. This flexible approach enables custom configurations that can accommodate complex surgeries, large open fields, and small, minimally invasive sites.

Some embodiments herein integrate the flexible tubes of the smoke evacuation system into the surgical drape, whether by attachment via clips and/or an adhesive or other method, or by direct integration of the flexible tubes into the surgical drape itself. By attaching or integrating the flexible tubes with the surgical drape's edges, the system maintains proximity to smoke generation zones. As the surgical drape is repositioned during surgical procedures, the flexible tube(s) move with it, ensuring continuous, source-aligned smoke capture.

Some existing approaches to smoke evacuation often encounter issues with noise, which can disrupt the operating environment. To combat this, some embodiments herein may include precisely angled holes within the flexible tubes to evacuate fluids. In some embodiments, the flexible tubes may include sound-dampening materials to further reduce turbulence and noise. Additional fluid flow optimization may also be incorporated into the design of the flexible tubes to maintain stable, laminar flow patterns, ensuring consistent capture efficiency and reduced distraction for surgical staff.

In order to combat the possible spread of pathogens via surgical smoke, some embodiments herein may employ medical-grade materials to construct the flexible tubes, fittings, and/or other components that are compatible with standard sterilization processes. Associated adhesives, mechanical clips, and optional integrated tubing (into the surgical drape) may also be designed according to similar standards and/or materials in order to maintain both sterility and secure attachment over time, even during sterilization procedures.

The embodiments herein may also be integrated with existing suction and medical waste infrastructure within an operating environment, which avoids time-consuming and expensive retrofits. This adaptable design supports the deployment of the embodiments herein in diverse healthcare settings, from resource-rich tertiary centers to smaller outpatient clinics.

### II. Example Systems

The following description and accompanying drawings will elucidate features of various example embodiments. The embodiments provided are by way of example, and are not intended to be limiting. As such, the dimensions of the drawings are not necessarily to scale.

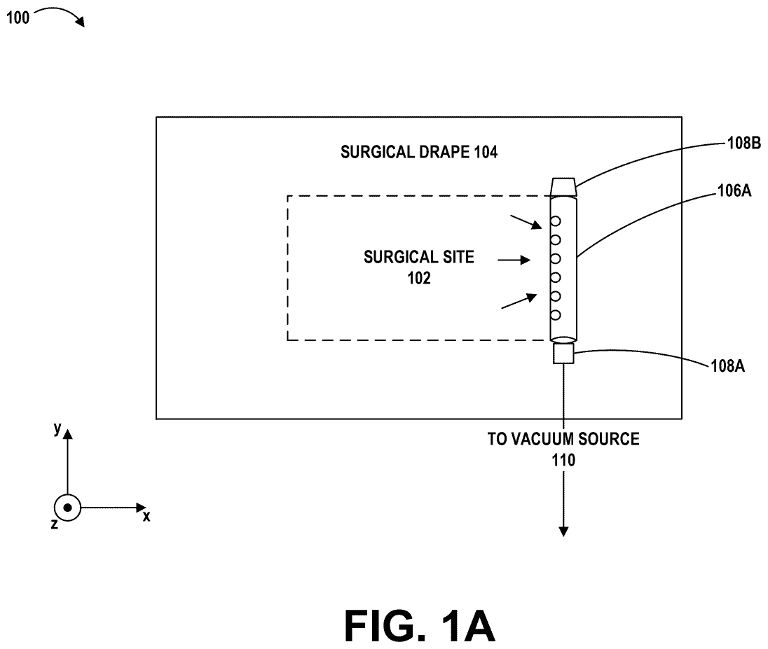

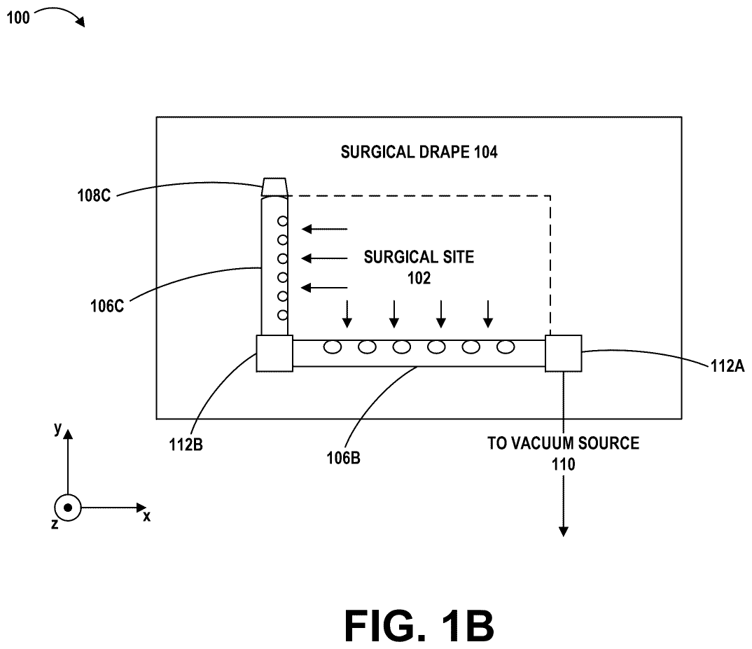

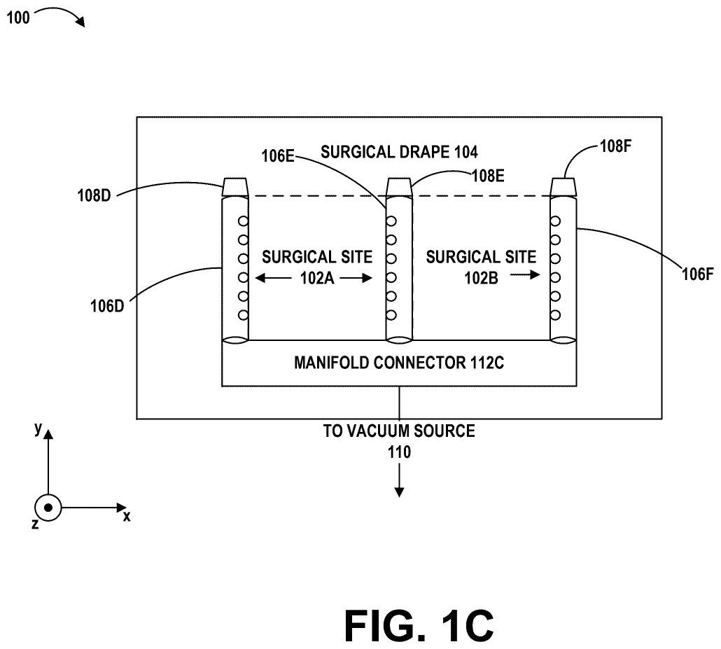

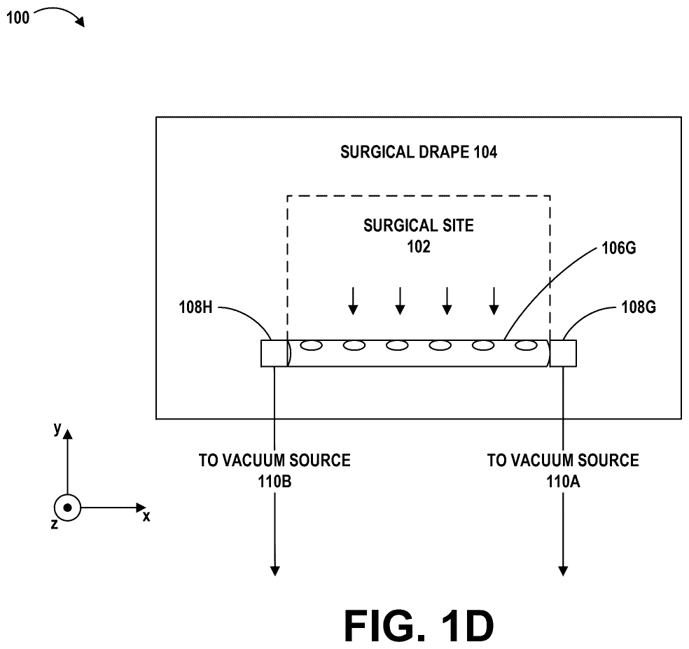

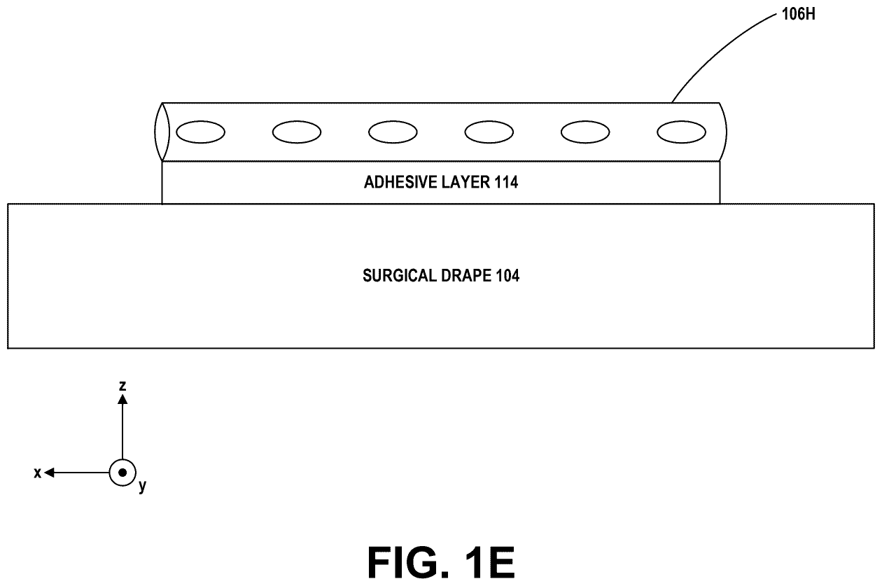

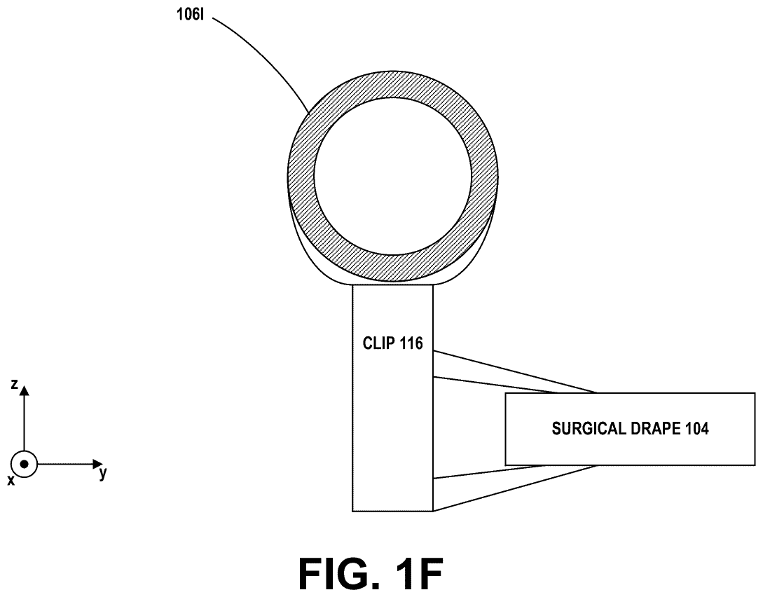

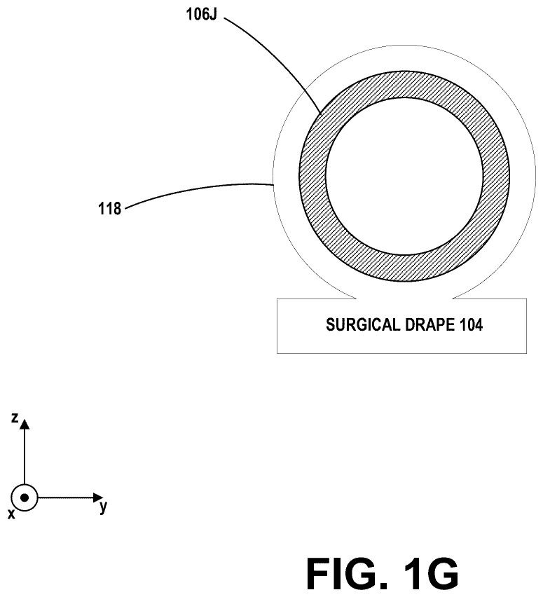

FIG. 1A is an illustration of an example system 100, according to example embodiments. The system 100 includes a surgical site 102 and a surgical drape 104. For instance, the surgical site 102 may be the area surrounding an incision made in a patient's body for a surgical intervention, such as an appendectomy (removal of the appendix) and/or types of thoracic surgeries. Thoracic surgeries involve interventions in the chest area, such as heart or lung procedures, and often produce larger amounts of surgical smoke than other surgical interventions. Surgical drapes such as the surgical drape 104 often serve to protect other areas of the patient's body during surgical procedures, as well as limit the spread of surgical disease through pathogens.

As shown in FIG. 1A, the system 100 may include one or more flexible tubes (e.g., the flexible tube 106A) positioned adjacent to at least one side of the surgical site 102. In this example, there is one flexible tube adjacent one side of the surgical site 102, but any number and/or arrangement of flexible tubes is possible in other embodiments, including 2, 3, 4, and/or 5 flexible tubes. Some of these alternative embodiments are described below with regard to FIGS. 1B, 1C, and 1D.

The flexible tube 106A may include a plurality of holes through which fluid may be evacuated (e.g., using suction generated by the vacuum source 110). This is shown in FIGS. 1A, 1B, and 1C with arrows showing the flow of fluid into the flexible tubes. The flexible tube 106A may be positioned in such a manner that the plurality of holes are directed towards (for instance, angled in a certain way) the surgical site 102, as shown in FIG. 1A. In some embodiments, the flexible tube 106A is positioned such that the plurality of holes are directed toward the surgical site at an angle of 15 to 90 degrees from a longitudinal axis of the flexible tube 106A (in the example of FIG. 1A, the longitudinal axis of the flexible tube 106A is understood as the axis parallel to the y-axis that runs along the length of the flexible tube 106A).

The flexible tube 106A may terminate at a fitting, as shown in FIG. 1A with fittings 108A and 108B. Herein, “fitting” refers to a connector or plug that may be connected to a flexible tube such that the tube is closed on an end and/or connected to other flexible tubes and/or other components. For instance, fitting 108A may be a connector for the flexible tube 106A to the vacuum source 110, while fitting 108B may be a plug for the ends of the flexible tube 106A that are not connected to another component. The fittings may be made of plastic, metal, or any other suitable material.

In some embodiments, one or more of the fittings may be lock fittings, which may be connectable to a vacuum source. For example, in FIG. 1A, fitting 108A is connected to a vacuum source 110, which is described in further detail below. For example, in some embodiments, a lock fitting may be used. A lock fitting is a type of fitting that allows for easy connection and disconnection for tubes. One example of such a lock fitting is a Luer lock. Some types of lock fittings may include a quick-release mechanism that allows for rapid attachment and detachment of the fitting from other components. Other types of lock fittings may include an integrated flow regulator, which, when used in a fitting connected to a vacuum source (e.g., fitting 108A connected to the vacuum source 110), may allow the flexible tubes connected to the fitting to have their fluid flow adjusted independently of other components of the system.

The system 100 may also include the vacuum source 110. The vacuum source may be connected to one or more fittings of the system 100, and may be configured to evacuate fluid generated at or near the surgical site 102 through the flexible tubes 106A by establishing a pressure differential. For instance, the vacuum source may generate pressure differential in a range of 25-750 mmHg. The vacuum source 110 may be a port on a wall of an operating room, a standalone machine or cart such as a STRYKER NEPTUNE or similar device, or integrated into other surgical equipment. Regardless, the fittings of the system 100 may be configured to be connected (directly or indirectly through other components) to the vacuum source 110 in any manner suitable for connection. In some embodiments, one or more of the flexible tubes may be connected to the vacuum source 110 on both ends, rather than just one as shown in FIG. 1A (e.g., as shown and described below with reference to FIG. 1D).

In some embodiments, the system 100 may include one or more manifold connectors that enable the one or more flexible tubes to be arranged in certain configurations, including series or parallel configurations. For instance, FIG. 1B depicts an embodiment of the system 100 with two flexible tubes 106B, 106C connected in a series configuration using manifold connectors 112A, 112B. Both of the flexible tubes 106B, 106C are thus connected to the vacuum source 110 by way of the manifold connector 112A, while one end of flexible tube 106C is plugged with fitting 108C.

FIG. 1C depicts an embodiment of the system 100 in a situation where there are multiple surgical sites 102A, 102B. In this example, a single manifold connector 112C is used with three flexible tubes 106D, 106E, 106F, each capped at one end by fittings 108D, 108E, 108F, respectively, where the flexible tubes 106D, 106E, 106F are arranged in a parallel configuration. This arrangement allows for adaptability in situations where there are either multiple surgical sites or multiple smoke emission points in larger surgical sites.

FIG. 1D depicts an embodiment of the system 100 with flexible tube 106G with fittings 108G, 108H on either end. The fittings 108G and 108H are connected to vacuum sources 110A and 110B, respectively. The fittings 108G, 108H may each include an integrated flow regulator, as described above, which allows for the flow of fluid at each end of the flexible tube 106G to be adjusted independently. For instance, if one portion of the surgical site 102 experiences higher smoke density, the corresponding end of the flexible tube 106G may have its fluid flow increased respectively. In some embodiments, fittings in this configuration (e.g., 108G and 108H) may be capped with removable seals, which may allow only one vacuum source to operate. The dual-vacuum source setup also allows for redundancy in the case that one source fails or if one end of the flexible tube 106G becomes obstructed.

During surgical procedures, the surgical drape 104 may be repositioned or moved, which may also impact the location and/or orientation of smoke evacuation devices. To address this, the flexible tube 106A of the system 100 may be removably attached to the surgical drape 104. This may be accomplished through different types of connection mechanisms, including an adhesive layer or strip (e.g., tape), one or more clips, and/or one or more mounts.

FIG. 1E depicts an example flexible tube 106H removably attached to the surgical drape 104 by way of an adhesive layer 114. The adhesive layer 114 may include any type of adhesive, such as tape (single or double-sided) or a liquid adhesive. In some embodiments, the adhesive layer 114 may be reusable across multiple surgeries. In other embodiments, the adhesive layer 114 may be disposable and replaceable in between surgeries.

In some embodiments, the one or more clips may include sleeve clips that encircle at least one of the one or more flexible tubes and are configured to attach to an edge of the surgical drape 104. Other clamps or clips may also be used, in some embodiments. FIG. 1F depicts an example flexible tube 106I (shown in cross-section) removably attached to the surgical drape 104 by way of clip 116, which clamps onto the surgical drape 104, while also cupping the flexible tube 106I.

In some embodiments, the flexible tubes may be directly integrated into the surgical drape 104 itself. An example of this is shown in FIG. 1G, where a flexible tube 106J (shown in cross-section) is threaded through a loop 118 defined within the surgical drape 104.

In some embodiments, the flexible tubes may be attached to the surgical drape 104 by way of flexible joints or connectors, which may allow the tube to bend and/or flex as the drape may move, thus maintaining the flexible tube's alignment with the surgical site 102. Depending on their configuration, such flexible connectors may permit a full range of motion for the flexible tubes. For instance, the flexible connectors may be formed of silicone and/or an elastic material that can be flexed repeatedly without experiencing degradation. As another example, the flexible connectors may include pivoting joints, which may also maintain the flexible tube's alignment with the surgical site 102 during motion of the surgical drape 104. Thus, by way of the system 100, smoke (or other fluid) from the surgical site 102 may, through suction, be evacuated from the surgical site 102, addressing many of the problems that such smoke can cause in surgical operating environments, as discussed above.

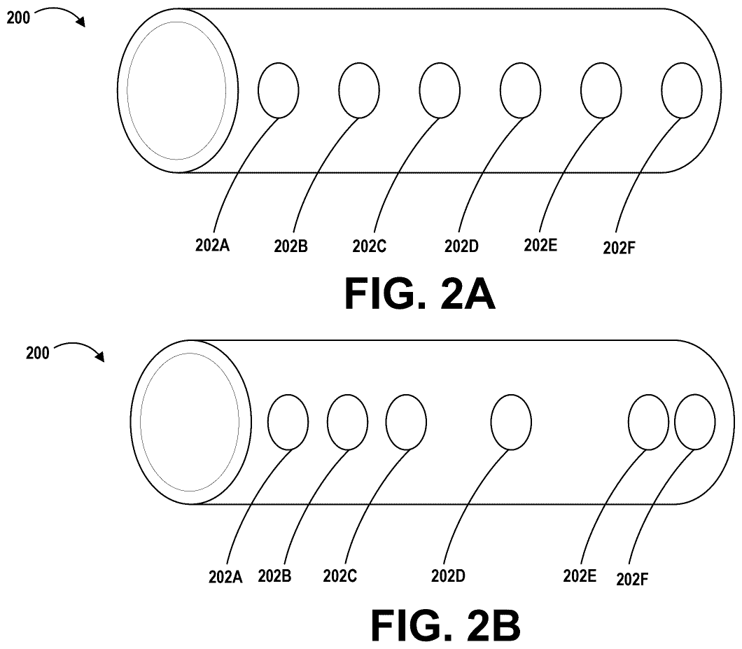

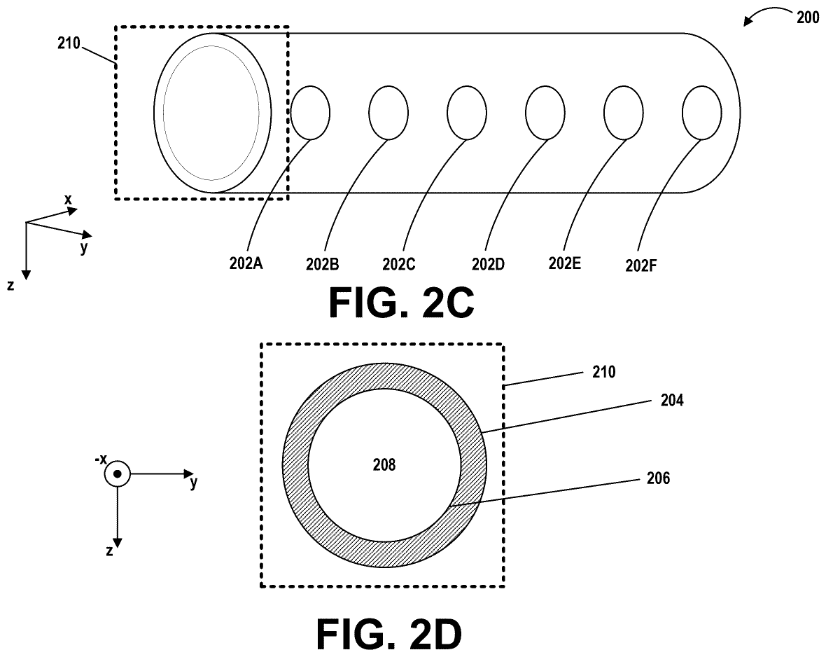

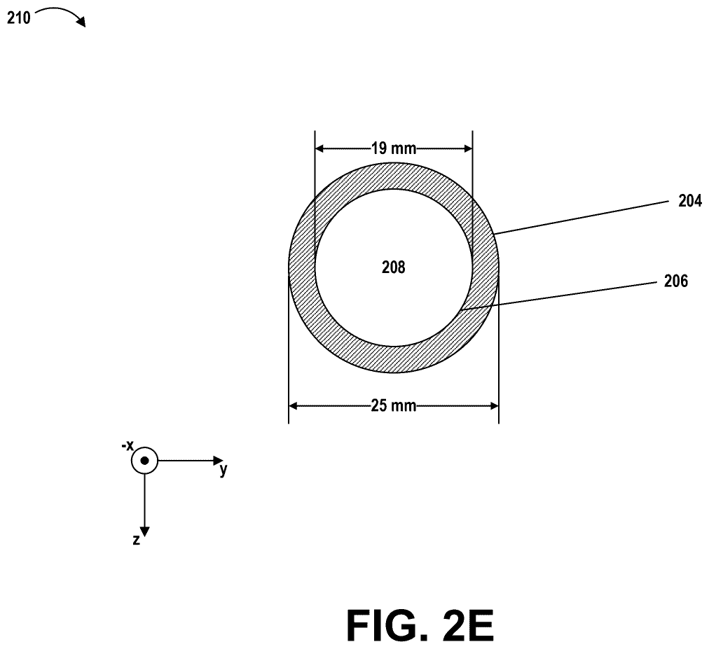

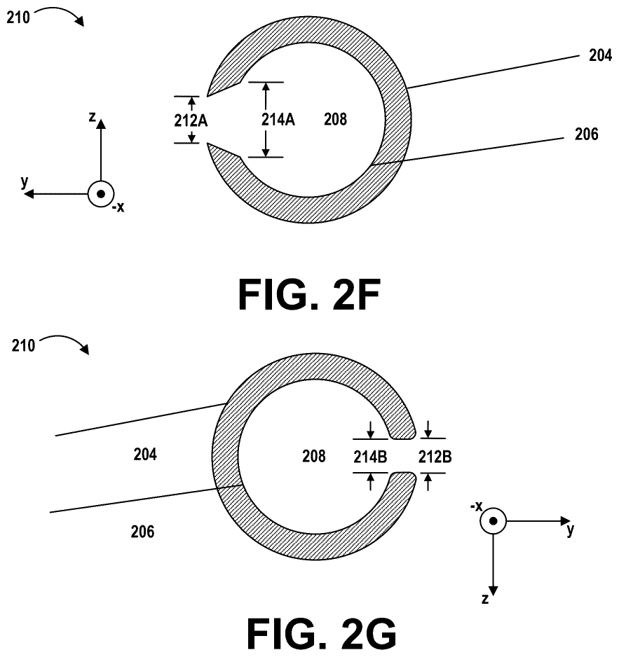

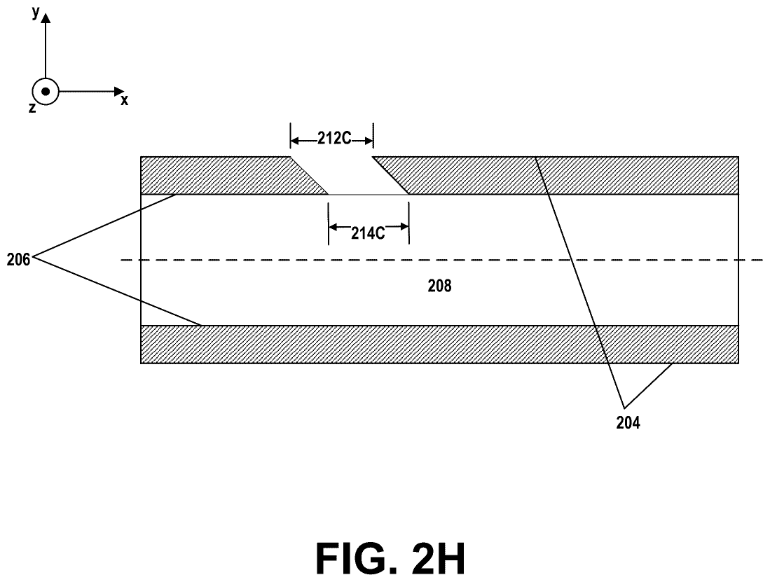

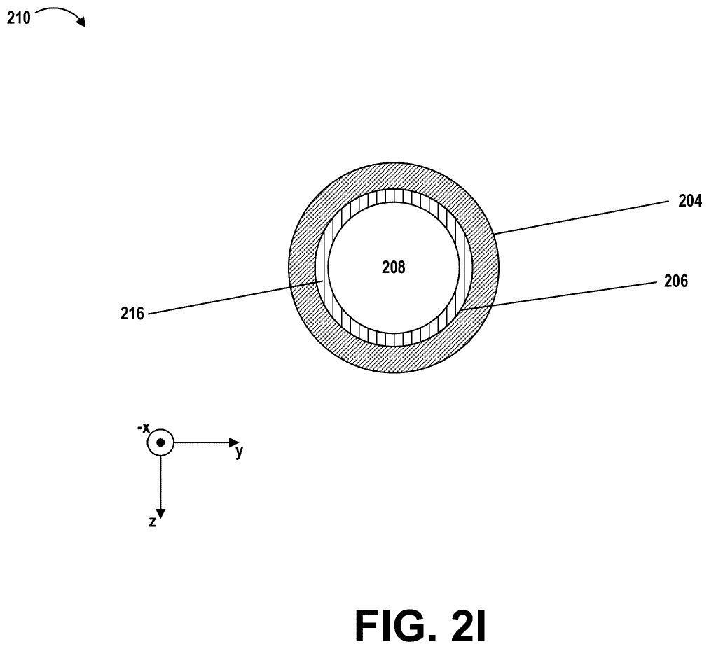

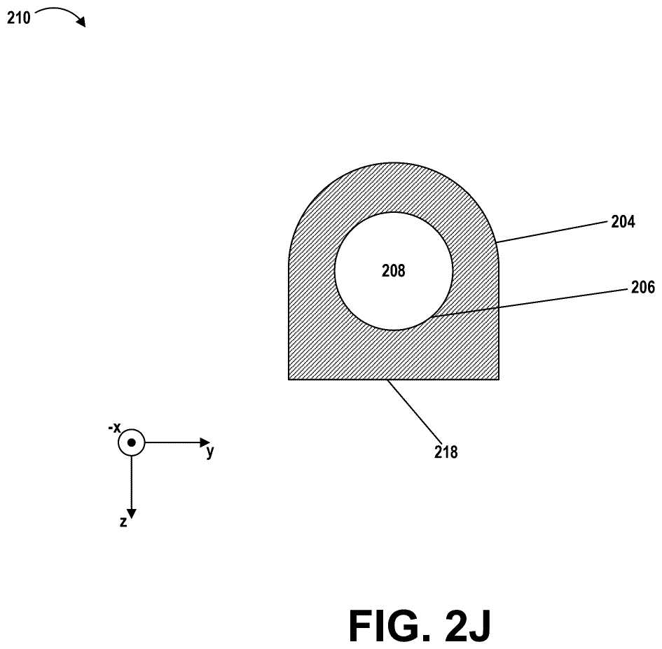

The flexible tubes and the plurality of holes therein are described in further detail with reference to FIGS. 2A-2J below. Generally, “longitudinal axis” in reference to FIGS. 2A-2J refers to an axis that runs lengthwise down the greater length of a flexible tube along the center of the flexible tube and parallel to the x-axis shown in each of FIGS. 2A-2J.

FIG. 2A is an illustration of an example flexible tube 200. The flexible tubes 106A, 106B, 106C, 106D, 106E, 106F, 106G, 106H, 106I, 106J in FIGS. 1A-1G may correspond to the flexible tube 200, for instance. The flexible tube 200 may be any length or configurable to be any length. For instance, the flexible tube 200 may be 10 -110 centimeters (e.g., 60 centimeters) in length.

The flexible tube 200 may be formed of several different types of materials, including extruded silicone (e.g., medical-grade silicone) and thermoplastic elastomer (TPE), among others. In some embodiments, the flexible tube 200 may be formed of a biodegradable material. This may allow the flexible tube 200 to be a single-use item, which may improve sterilization.

As shown, the flexible tube 200 may include a plurality of holes, including holes 202A, 202B, 202C, 202D, 202E, 202F. In some embodiments, as in FIG. 2A, the holes 202A, 202B, 202C, 202D, 202E, 202F are each evenly spaced along an exterior surface of the flexible tube 200. In other words, the plurality of holes may be evenly spaced along exterior surfaces of each of the respective one or more flexible tubes.

However, in other embodiments, the holes 202A, 202B, 202C, 202D, 202E, and 202F are each variably spaced along the exterior surface of the flexible tube 200, as shown in an alternative configuration of the flexible tube 200 in FIG. 2B. As shown, holes 202A, 202B, and 202C are spaced closer together than holes 202C and 202D. This may be done for several reasons; for instance, the variable spacing may be done according to expected fluid densities that vary across different regions of the surgical site (e.g., holes may be more densely spaced at regions within a surgical site that are more likely to emit smoke and/or that typically emit greater quantities of smoke). Using the example of FIG. 2B, holes 202A, 202B, 202C may be positioned near a region of a surgical site expected to produce greater quantities of smoke, while a region with lesser quantities of smoke may be aligned with hole 202D. In other words, the plurality of holes are variably spaced along exterior surfaces of each of the respective one or more flexible tubes according to expected fluid densities emitted from associated regions of the surgical site. In some embodiments, the minimum spacing between each hole of the plurality of holes 202A, 202B, 202C, 202D, 202E, and 202F may be between 4 and 12 millimeters (e.g., 8 millimeters).

Each hole of the holes 202A, 202B, 202C, 202D, 202E, and 202F may have the same or a different diameter. For instance, each hole may have a diameter defined at the exterior surface of the flexible tube 200 of between 0.5 millimeters and 5 millimeters (e.g., 1 millimeter, 2 millimeters, 3 millimeters, or 4 millimeters).

FIG. 2C shows another view of the flexible tube 200, while FIG. 2D shows a cross-sectional view 210 of the flexible tube 200. As shown, the flexible tube 200 includes an exterior surface 204 (as discussed above) and an interior surface 206. The interior surface 206 surrounds an interior chamber 208 within the flexible tube 200.

The diameter of the flexible tube 200 at both the exterior surface 204 and the interior surface 206 may vary (e.g., along the length of a single flexible tube or across different flexible tubes). For instance, the diameter of the interior surface 206 (and thus the interior chamber 208) may be between 5 millimeters and 30 millimeters and the diameter of the exterior surface 204 may be between 5 millimeters and 100 millimeters. This is shown in FIG. 2E, where the interior surface 206 has a diameter of 19 millimeters and the exterior surface 204 has a diameter of 25 millimeters. These measurements are provided as examples; other values are possible in other embodiments.

The holes 202A, 202B, 202C, 202D, 202E, 202F may be defined between the exterior surface 204 and the interior surface 206 such that the interior chamber 208 is fluidly connected to an exterior of the flexible tube, as shown in FIGS. 2F and 2G. In some embodiments, each of the holes 202A, 202B, 202C, 202D, 202E, 202F may have a first diameter 212A defined in the exterior surface 204 and a second diameter 214A defined in the interior surface 206. This allows for variations in the shapes of the holes. For instance, the first diameter 212A may be smaller than the second diameter 214A, which may result in a hole having a fluted shape, as shown in FIG. 2F where the first diameter 212A is smaller than the second diameter 214A. This, in turn, may affect the vibrational frequency or the intake velocity of the fluid when the flexible tube 200 is in use, which may affect the amount of noise generated.

FIG. 2G depicts the hole of the holes 202A, 202B, 202C, 202D, 202E, 202F where the first diameter 212B and the second diameter 214B are the same. FIGS. 2F and 2G depict a situation where both the first diameter 212A, 212B and second diameter 214A, 214B extend radially from the center of the tube in the same plane/at a right angle.

FIG. 2H depicts another cross-sectional view of a flexible tube. This view represents a “cutaway” view of the flexible tube (i.e., a cross section of the flexible tube taken along a plane parallel to the x-z plane). In this example, the hole of the holes 202A, 202B, 202C, 202D, 202E, 202F is depicted as angled with regard to the longitudinal axis of the tube (represented in FIG. 2H as the dashed line). In this example, the first diameter 212C and second diameter 214C are offset from each other due to the angled shape of the hole between the exterior surface 204 and the interior surface 206.

The shape of each of the holes 202A, 202B, 202C, 202D, 202E, 202F may vary based on different conditions or considerations. For instance, each hole may have chamfered or rounded edges, which may reduce turbulence and/or noise during suction. As an example, FIGS. 2F and 2H show the hole with a chamfered edge, while FIG. 2G shows the hole with a rounded edge.

Noise produced by the flexible tube 200 may also be addressed in other ways. In some embodiments, a lining 216 may be disposed on the interior surface 206 of the flexible tube 200, as shown in FIG. 2I. The lining 216 may include a sound-dampening material. Example sound-dampening materials include open-cell foam and/or other porous materials. In some embodiments, the lining 216 may include a detachable sleeve that may line the interior surface 206. The interior surface 206 may also be formed in a specific way, based on maximum flow rate and/or pressure, in order to minimize noise.

The flexible tube 200 may also be configured to eradicate pathogens that may be present in fluids flowing through the respective flexible tube 200 during fluid transport. In some embodiments, for example, the lining 216 may include an antimicrobial coating. The flexible tube 200, depending on the material(s) it is formed of, may be compatible with several sterilization methods, including autoclaving, ethylene oxide sterilization, and hydrogen peroxide plasma sterilization.

In some embodiments, the exterior surface 204 may include a flattened surface that runs longitudinally along a length of the respective flexible tube 200. In other words, a portion of the exterior surface 204 may be flat in order to provide greater stability to the flexible tube 200 while in use. For instance, the flattened surface may help prevent the flexible tube 200 from inadvertently rolling away from (or rolling such that the holes 202A, 202B, 202C, 202D, 202E, 202F are no longer angled properly with respect to) the surgical site 102. This is shown in FIG. 2J, which is a cross-section of a flexible tube with a flattened surface 218 as one portion of the exterior surface 204.

In some embodiments, the exterior surface 204 may include visual indicators that may aid in the placement of the flexible tube 200 with regard to the surgical drape 104. For instance, high, medium, and low-density smoke zones may be color-coded. In some embodiments, the exterior surface 204 may include reflective markings that may aid visibility in low-light surgical environments. In some embodiments, the exterior surface 204 may include markings associated with specific anatomical landmarks related to the surgical site 102. Likewise, in some embodiments, the surgical drape may include one or more fiducial markings to assist with alignment and attachment of the flexible tube 200. Such fiducial markings on the surgical drape 104 may correspond with respective fiducial markings on the exterior surface 204 of the flexible tube 200, for example.

Following the above, an example apparatus may include a flexible tube (e.g., flexible tube 200) including an exterior surface (e.g., exterior surface 204) surface and an interior surface (e.g., interior surface 206). The interior surface surrounds an interior chamber, and one or more holes (e.g., the holes 202A, 202B, 202C, 202D, 202E, 202F) are defined between the exterior surface and the interior surface of the flexible tube such that the interior chamber (e.g., interior chamber 208) is fluidly connected to an exterior of the flexible tube. The flexible tube terminates at a fitting (e.g., fitting 108A) configured to connect to a vacuum source (e.g., vacuum source 110) in order to evacuate fluid generated at a surgical site (e.g., surgical site 102) through the one or more holes via a pressure differential. The example apparatus may also include an adhesive strip, one or more clips, or one or more mounts connected to the flexible tube and configured to removably attach the flexible tube to a surgical drape (e.g., surgical drape 104).

In some embodiments, different configurations of flexible tubes (with different dimensions, materials, hole size, hole spacing, and/or hole angle) may be used for different types of surgeries or other procedures, and/or to interface with different types of vacuum sources.

### III. Example Methods

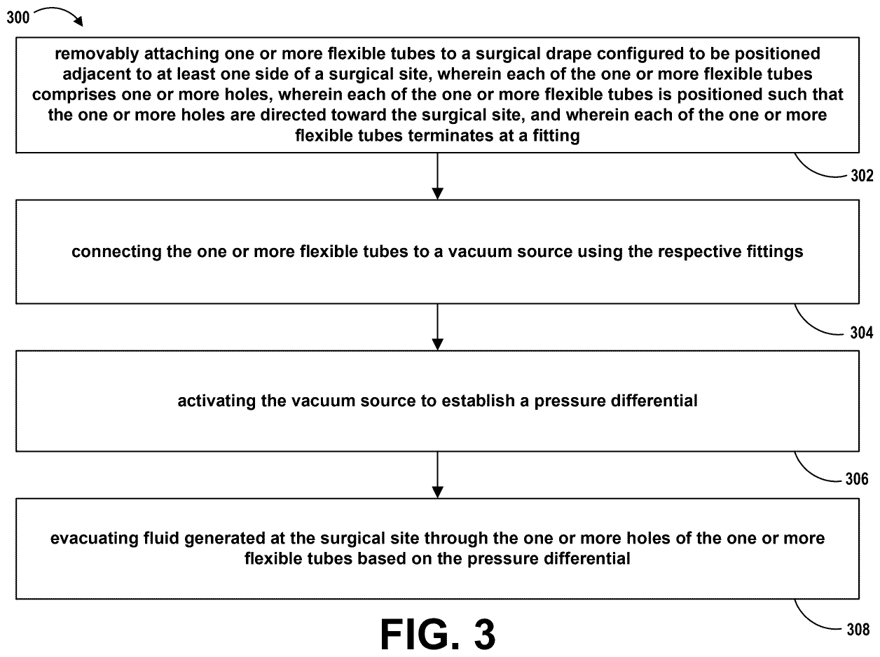

FIG. 3 is a flow chart illustrating an example method 300. The method 300 illustrated by FIG. 3 may be carried out by or in relation to a system (e.g., the system 100 and/or the apparatus described above). The embodiments of FIG. 3 may be simplified by the removal of any one or more of the features shown therein. Further, these embodiments may be combined with features, aspects, and/or implementations of any of the previous figures or otherwise described herein.

Block 302 may involve removably attaching one or more flexible tubes to a surgical drape configured to be positioned adjacent to at least one side of a surgical site, wherein each of the one or more flexible tubes comprises one or more holes, wherein each of the one or more flexible tubes is positioned such that the one or more holes are directed toward the surgical site, and wherein each of the one or more flexible tubes terminates at a fitting.

Block 304 may involve connecting the one or more flexible tubes to a vacuum source using the respective fittings.

Block 306 may involve activating the vacuum source to establish a pressure differential.

Block 308 may involve evacuating fluid generated at the surgical site through the one or more holes of the one or more flexible tubes based on the pressure differential.

### IV. Conclusion

The present disclosure is not to be limited in terms of the particular embodiments described in this application, which are intended as illustrations of various aspects. Many modifications and variations can be made without departing from its scope, as will be apparent to those skilled in the art. Functionally equivalent methods and apparatuses within the scope of the disclosure, in addition to those described herein, will be apparent to those skilled in the art from the foregoing descriptions. Such modifications and variations are intended to fall within the scope of the appended claims.

The above detailed description describes various features and operations of the disclosed systems, devices, and methods with reference to the accompanying figures. The example embodiments described herein are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations.

The particular arrangements shown in the figures should not be viewed as limiting. It should be understood that other embodiments can include more or less of each element shown in a given figure. Further, some of the illustrated elements can be combined or omitted. Yet further, an example embodiment can include elements that are not illustrated in the figures.

While various aspects and embodiments have been disclosed herein, other aspects and embodiments will be apparent to those skilled in the art. The various aspects and embodiments disclosed herein are for purpose of illustration and are not intended to be limiting, with the true scope being indicated by the following claims.

What is claimed is:

1. A system comprising:
a surgical drape configured to be positioned adjacent to at least one side of a surgical site;
one or more flexible tubes removably attached to or positioned within the surgical drape, wherein each of the one or more flexible tubes comprises a plurality of holes, wherein each of the one or more flexible tubes is positioned such that the plurality of holes are directed toward the surgical site, and wherein each of the one or more flexible tubes terminates at a fitting; and
a vacuum source connected to the respective fittings of the one or more flexible tubes and configured to evacuate fluid generated at the surgical site through the plurality of holes by establishing a pressure differential.

2. The system of claim 1, further comprising one or more manifold connectors enabling the one or more flexible tubes to be arranged in a series or parallel configuration.

3. The system of claim 1, wherein the one or more flexible tubes are removably attached to the surgical drape by an adhesive strip, one or more clips, or one or more mounts.

4. The system of claim 3, wherein the one or more clips comprise sleeve clips that encircle at least one of the one or more flexible tubes and are configured to attach to an edge of the surgical drape.

5. The system of claim 1, wherein the plurality of holes are evenly spaced along exterior surfaces of each of the respective one or more flexible tubes.

6. The system of claim 1, wherein the plurality of holes are variably spaced along exterior surfaces of each of the respective one or more flexible tubes according to expected fluid densities emitted from associated regions of the surgical site.

7. The system of claim 1, wherein each hole of the plurality of holes has a diameter defined at an exterior surface of the respective flexible tube of 0.5 millimeters to 5 millimeters.

8. The system of claim 1, wherein each of the one or more flexible tubes comprises an exterior surface and an interior surface, wherein the interior surface surrounds an interior chamber, wherein the plurality of holes are defined between the exterior surface and the interior surface of the respective flexible tube such that the interior chamber is fluidly connected to an exterior of the flexible tube.

9. The system of claim 8, wherein a diameter of the interior chamber is 5 millimeters to 30 millimeters, and wherein a diameter of the exterior surface is 5 millimeters to 100 millimeters.

10. The system of claim 8, wherein the exterior surface comprises a flattened surface that runs longitudinally along a length of the respective flexible tube.

11. The system of claim 8, wherein each of the plurality of holes has a first diameter defined in the exterior surface and a second diameter defined in the interior surface, and wherein the first diameter is smaller than the second diameter.

12. The system of claim 8, wherein a sound-dampening material lines the interior surface of each of the flexible tubes.

13. The system of claim 8, wherein an antimicrobial coating is disposed on the interior surface.

14. The system of claim 1, wherein each of the one or more flexible tubes is positioned such that the plurality of holes are directed toward the surgical site at an angle of 15 to 90 degrees from a longitudinal axis of the tube.

15. The system of claim 1, wherein each of the one or more flexible tubes comprises extruded silicone or thermoplastic elastomer (TPE).

16. The system of claim 1, wherein the fittings of the one or more flexible tubes are lock fittings, and wherein the lock fittings are connectable to the vacuum source.

17. The system of claim 16, wherein each lock fitting comprises an integrated flow regulator.

18. The system of claim 1, wherein each of the one or more flexible tubes comprises a biodegradable material.

19. A method comprising:
removably attaching one or more flexible tubes to a surgical drape configured to be positioned adjacent to at least one side of a surgical site, wherein each of the one or more flexible tubes comprises one or more holes, wherein each of the one or more flexible tubes is positioned such that the one or more holes are directed toward the surgical site, and wherein each of the one or more flexible tubes terminates at a fitting;
connecting the one or more flexible tubes to a vacuum source using the respective fittings;
activating the vacuum source to establish a pressure differential; and
evacuating fluid generated at the surgical site through the one or more holes of the one or more flexible tubes based on the pressure differential.

20. An apparatus comprising:
a flexible tube comprising an exterior surface and an interior surface, wherein the interior surface surrounds an interior chamber, wherein one or more holes are defined between the exterior surface and the interior surface of the flexible tube such that the interior chamber is fluidly connected to an exterior of the flexible tube, and wherein the flexible tube terminates at a fitting configured to connect to a vacuum source in order to evacuate fluid generated at a surgical site through the one or more holes via a pressure differential; and
an adhesive strip, one or more clips, or one or more mounts connected to the flexible tube and configured to removably attach the flexible tube to a surgical drape.
